(19) United States
(12) Patent Application Publication
(10) Pub. No.: US 2004/0209262 A1
(43) Pub. Date: Oct. 21, 2004

(54) Biopolymeric arrays comprising test probes for two or more different species and methods for using the same

(76) Inventors: Jay K. Bass, Kennett Square, PA (US); Theodore R. Sana, San Mateo, CA (US); Sean J. Coughlan, Hockessin, DE (US)

Correspondence Address: AGILENT TECHNOLOGIES, INC., INTELLECTUAL PROPERTY ADMINISTRATION, LEGAL DEPT., P.O. BOX 7599, M/S DL429, LOVELAND, CO, 80537-0599, US

(21) Appl. No.: 10/420,575

(22) Filed: Apr. 21, 2003

(51) Int. Cl. C12Q 1/68

(52) U.S. Cl. 435/6

(57) ABSTRACT

Biopolymeric arrays, e.g., nucleic acid and peptide arrays, and methods for using the same are provided. A feature of the subject arrays is that they include test probes for two or more different species, where the species may have a known interaction, e.g., they may have a known parasite/host or pathogen/host relationship. Also provided are method of using the subject arrays, e.g., in assays where the interaction of two or more different species at the genomic and/or protein levels is evaluated. The subject arrays and methods of using the same find use in a variety of different applications.

### FIELD OF THE INVENTION

The present invention relates to biopolymeric arrays.

### BACKGROUND OF THE INVENTION

Array assays between surface bound binding agents or probes and target molecules in solution may be used to detect the presence of particular biopolymeric analytes in the solution. The surface-bound probes may be nucleic acids (e.g., oligonucleotides, polynucleotides), peptides (e.g., polypeptides, proteins, antibodies) or other molecules capable of binding with target biomolecules in the solution(i.e. glycans). Such binding interactions are the basis for many of the methods and devices used in a variety of different fields, e.g., genomics (in sequencing by hybridization, SNP detection, differential gene expression analysis, identification of novel genes, gene mapping, finger printing, etc.) and proteomics.

One typical array assay method involves biopolymeric probes immobilized in an array on a surface of a substrate such as a glass substrate or the like. A solution containing target molecules (“targets”) that bind with the attached probes is placed in contact with the bound probes under conditions sufficient to promote binding of targets in the solution to the complementary probes on the substrate to form a binding complex that is bound to the surface of the substrate. The pattern of binding by target molecules to probe features or spots on the substrate produces a pattern, i.e., a binding complex pattern, on the surface of the substrate which is detected. This detection of binding complexes provides desired information about the target biomolecules in the solution.

The binding complexes may be detected by reading or scanning the array with, for example, optical means, although other methods may also be used, as appropriate for the particular assay. For example, laser light may be used to excite fluorescent labels attached to the targets, generating a signal only in those spots on the array that have a labeled target molecule bound to a probe molecule. This pattern may then be digitally scanned for computer analysis. Such patterns can be used to generate data for biological assays such as the identification of drug targets, single-nucleotide polymorphism mapping, monitoring samples from patients to track their response to treatment, assessing the efficacy of new treatments, etc.

In applications where one wishes to compare the interactions of two or more species, currently two separate array assays must be performed, one for each species. The present invention provides an improved way for practicing such array-based assays in which the interaction between 2 or more species, e.g., at the genomic or proteomic levels, is evaluated.

Relevant Literature

U.S. Pat. No. 6,177,248. See also Wang et al., “Microarray-based detection and genotyping of viral pathogens,” Proc. Nat'l Acad. Sci. USA (Nov. 26, 2002) 99:15687-15692.

### SUMMARY OF THE INVENTION

Biopolymeric arrays, e.g., nucleic acid and peptide arrays, and methods for using the same are provided. A feature of the subject arrays is that they include test probes for two or more different species, where the species may have a known interaction, e.g., they may have a known parasite/host or pathogen/host relationship. Also provided are method of using the subject arrays, e.g., in assays where the interaction of two or more different species at the genomic and/or protein levels is evaluated. The subject arrays and methods of using the same find use in a variety of different applications.

### BRIEF DESCRIPTION OF THE FIGURES

FIG. 1 shows an exemplary substrate carrying an array, such as may be used in the devices of the subject invention.

FIG. 2 shows an enlarged view of a portion of FIG. 1 showing spots or features.

FIG. 3 is an enlarged view of a portion of the substrate of FIG. 2.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The term “biomolecule” means any organic or biochemical molecule, group or species of interest that may be formed in an array on a substrate surface. Exemplary biomolecules include peptides, proteins, amino acids and nucleic acids.

The term “peptide” as used herein refers to any compound produced by amide formation between a carboxyl group of one amino acid and an amino group of another group.

The term “oligopeptide” as used herein refers to peptides with fewer than about 10 to 20 residues, i.e. amino acid monomeric units.

The term “polypeptide” as used herein refers to peptides with more than 10 to 20 residues.

The term “protein” as used herein refers to polypeptides of specific sequence of more than about 50 residues.

The term “nucleic acid” as used herein means a polymer composed of nucleotides, e.g., deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “nucleoside” and “nucleotide” are intended to include those moieties which contain not only the known purine and pyrimidine base moieties, but also other heterocyclic base moieties that have been modified. Such modifications include methylated purines or pyrimidines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “ribonucleic acid” and “RNA” as used herein refer to a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The term “polynucleotide” as used herein refers to single or double stranded polymer composed of nucleotide monomers of generally greater than 100 nucleotides in length.

A “biopolymer” is a polymeric biomolecule of one or more types of repeating units. Biopolymers are typically found in biological systems and particularly include polysaccharides (such as carbohydrates), peptides (which term is used to include polypeptides and proteins) and polynucleotides as well as their analogs such as those compounds composed of or containing amino acid analogs or non-amino acid groups, or nucleotide analogs or non-nucleotide groups.

A “biomonomer” references a single unit, which can be linked with the same or other biomonomers to form a biopolymer (e.g., a single amino acid or nucleotide with two linking groups, one or both of which may have removable protecting groups).

An “array,” includes any one-dimensional, two-dimensional or substantially two-dimensional (as well as a three-dimensional) arrangement of addressable regions bearing a particular chemical moiety or moieties (e.g., biopolymers such as polynucleotide or oligonucleotide sequences (nucleic acids), polypeptides (e.g., proteins), carbohydrates, lipids, etc.) associated with that region. In the broadest sense, the preferred arrays are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein.

With respect to methods in which premade probes are immobilized on a substrate surface, immobilization of the probe to a suitable substrate may be performed using conventional techniques. See, e.g., Letsinger et al. (1975) Nucl. Acids Res. 2:773-786; Pease, A. C. et al., Proc. Nat. Acad. Sci. USA, 1994, 91:5022-5026. The surface of a substrate may be treated with an organosilane coupling agent to functionalize the surface. One exemplary organosilane coupling agent is represented by the formula RₙSiY(₄₋ₙ) wherein: Y represents a hydrolyzable group, e.g., alkoxy, typically lower alkoxy, acyloxy, lower acyloxy, amine, halogen, typically chlorine, or the like; R represents a nonhydrolyzable organic radical that possesses a functionality which enables the coupling agent to bond with organic resins and polymers; and n is 1, 2 or 3, usually 1. One example of such an organosilane coupling agent is 3-glycidoxypropyltrimethoxysilane (“GOPS”), the coupling chemistry of which is well-known in the art. See, e.g., Arkins, “Silane Coupling Agent Chemistry,” Petrarch Systems Register and Review, Eds. Anderson et al. (1987). Other examples of organosilane coupling agents are (γ-aminopropyl)triethoxysilane and (γ-aminopropyl)trimethoxysilane. Still other suitable coupling agents are well known to those skilled in the art. Thus, once the organosilane coupling agent has been covalently attached to the support surface, the agent may be derivatized, if necessary, to provide for surface functional groups. In this manner, support surfaces may be coated with functional groups such as amino, carboxyl, hydroxyl, epoxy, aldehyde and the like.

Use of the above-functionalized coatings on a solid support provides a means for selectively attaching probes to the support. For example, an oligonucleotide probe formed as described above may be provided with a 5′-terminal amino group which can be reacted to form an amide bond with a surface carboxyl using carbodiimide coupling agents. 5′ attachment of the oligonucleotide may also be effected using surface hydroxyl groups activated with cyanogen bromide to react with 5′-terminal amino groups. 3′-terminal attachment of an oligonucleotide probe may be effected using, for example, a hydroxyl or protected hydroxyl surface functionality.

Also, instead of drop deposition methods, light directed fabrication methods may be used, as are known in the art. Inter-feature areas need not be present particularly when the arrays are made by light directed synthesis protocols.

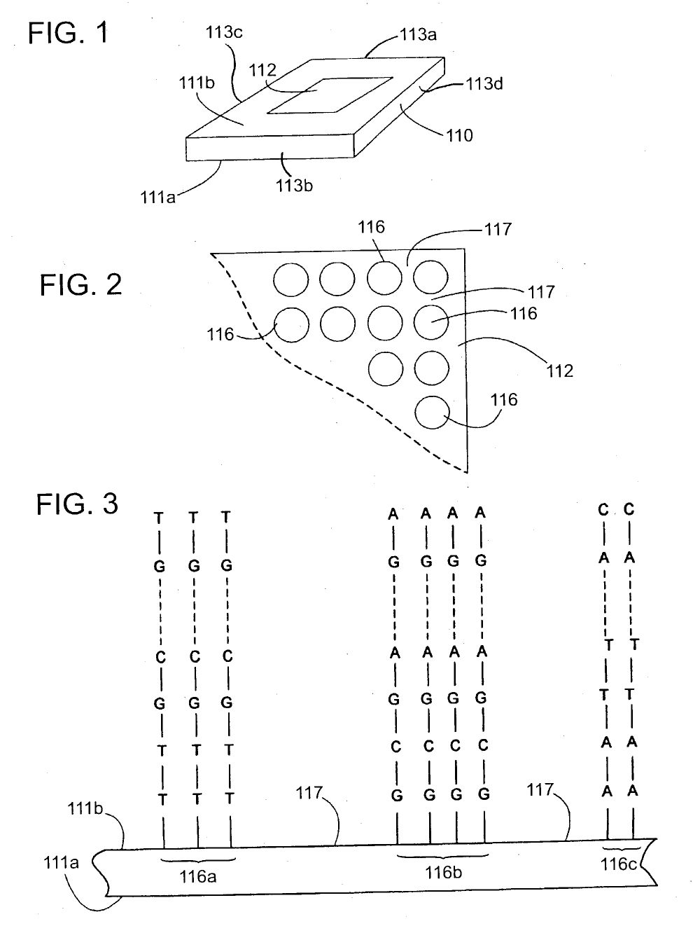

An exemplary array is shown in FIGS. 1-3, where the array shown in this representative embodiment includes a contiguous planar substrate 110 carrying an array 112 disposed on a rear surface 111b of substrate 110. It will be appreciated though, that more than one array (any of which are the same or different) may be present on rear surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymers, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on front surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

The term “flexible” is used herein to refer to a structure, e.g., a bottom surface or a cover, that is capable of being bent, folded or similarly manipulated without breakage. For example, a cover is flexible if it is capable of being peeled away from the bottom surface without breakage.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

The substrate may be flexible (such as a flexible web). When the substrate is flexible, it may be of various lengths including at least 1 m, at least 2 m, or at least 5 m (or even at least 10 m).

The term “rigid” is used herein to refer to a structure, e.g., a bottom surface or a cover that does not readily bend without breakage, i.e., the structure is not flexible.

The term “stringent hybridization conditions” as used herein refers to conditions that are compatible to produce duplexes on an array surface between complementary binding members, i.e., between probes and complementary targets in a sample, e.g., duplexes of nucleic acid probes, such as DNA probes, and their corresponding nucleic acid targets that are present in the sample, e.g., their corresponding mRNA analytes present in the sample. An example of stringent hybridization conditions is hybridization at 60° C. or higher and 3×SSC (450 mM sodium chloride/45 mM sodium citrate). Another example of stringent hybridization conditions is incubation at 42° C. in a solution containing 30% formamide, 1M NaCl, 0.5% sodium sarcosine, 50 mM MES, pH 6.5. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

The term “sample” as used herein relates to a material or mixture of materials, typically, although not necessarily, in fluid form, containing one or more components of interest.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

### DETAILED DESCRIPTION OF THE INVENTION

Biopolymeric arrays, e.g., nucleic acid and peptide arrays, and methods for using the same are provided. A feature of the subject arrays is that they include test probes for two or more different species, where the species may have a known interaction, e.g., they may have a known parasite/host or pathogen/host relationship. Also provided are method of using the subject arrays, e.g., in assays where the interaction of two or more different species at the genomic and/or protein levels is evaluated. The subject arrays and methods of using the same find use in a variety of different applications.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components that are described in the publications that might be used in connection with the presently described invention.

In further describing the invention in greater detail than provided in the Summary and as informed by the Background and Definitions provided above, the subject arrays are described first in greater detail, followed by a review of representative methods for their fabrication. Next, a description of representative applications in which the subject arrays find use is provided, as well as a review of representative kits that include the arrays and find use applications thereof.

Arrays

As indicated above, the subject invention provides arrays of biomolecules, e.g., arrays of biopolymers. More specifically, the arrays are arrays of features, where each feature comprises a biomolecule, as described above, e.g., nucleic acids, peptides, etc. All of the distinct features of the array may be different, or features may be present in multiple copies, e.g., in duplicate or in triplicate, etc.

The arrays are characterized by including test probes two or more different species, i.e., test probes for targets from two or more different species. The term “species” is used broadly and refers to the smallest taxonomical grouping of any of archaea, bacteria, eukaryota, viroids and viruses. Any two species are distinct or different if they are not taxonomically grouped as, i.e., considered to be, the same. Any two different or distinct species may be from different taxonomical kingdoms, or from within the same kingdom, class, order, phylum, or genus, so long as they are not of the same species. The arrays may contain test probes for two different species, or more than two different species, such as three, four, five or more different species, where the total number of different species represented on a given array is typically not more than about 10, e.g., not more than about 7.

By “test probe” is meant a probe molecule that specifically binds to a target during use of the array. The test probe is immobilized on the surface of a solid support and binds to a target in a fluid upon contact of the fluid with the surface bearing the test probe. The test probes of the subject arrays are not “control” probes, which are probes that are directed to non-target entities which might be present in the fluid contacted with the array surface during use. Control probes or features thereof are well known to those of skill in the art, and include probes or features directed to contaminants, normalization probes/features, etc. Since a test probes is not a control probe, it is a probe that specifically binds to a target analyte, where the analyte is an analyte of specific interest to a given assay, i.e., an analyte that characterizes the assay. As pointed out in the definitions section, above, either of the targets or probes may be the unknown entities that are being evaluated by the other, depending on the particular assay being performed.

As mentioned above, the subject arrays include test probes or features thereof for two or more different species. As the arrays include test probes or features for two or more different species, the total population of test probes on the subject arrays can be divided into two or more subsets, where each subset is made up of probes (features thereof) directed to a different or distinct species. In other words, the subject arrays include at least a first set of test probes/features that specifically bind to targets from a first species and a second set of test probes/features that specifically bind to targets from a second species. Said another way, in the total collection or sum of different probes or features of the array, there is at least a first group that specifically binds (e.g., by hybridizing to target analytes or binding to target proteins, etc.) to targets from a first species and a second group that specifically binds to target analytes from a second species.

With respect to the relative numbers of the different groups or sets of features for a given array, in certain embodiments the number ratio of probes/features for any two sets of probes/features for a given array ranges from about 1/10 to 1, including from about 1/5, such as from about 1/3 or 1/2 or 1/1. In certain embodiments, the number of target probes for any given species is at least about 5, such as at least about 10, at least about 25, at least about 50, at least about 100, at least about 250, at least about 500, at least about 1000, at least about 5000 or more, such as at least about 10,000 or more.

The arrays of the present invention may have a number of different configurations. For example, in certain embodiments the arrays may include a single substrate having a surface displaying all of the probes or features of the array. In yet other embodiments, the array may be made up of a plurality of substrates or solid supports, e.g., small beads or particles, where typically in such embodiments, each individual solid support or substrate displays a different probe on its surface, such that each different support or substrate is a different feature of the array, e.g., as found in a “fluid” or “liquid” array, which formats are well known to those of skill in the art.

In those embodiments where the array is made up of a single solid support, the array typically includes a planar surface on which the different biomolecular probes/features are displayed, as described in greater detail above. In such embodiments, the subsets or groups of features that correspond to different species may be arranged relative to each other on the surface of the substrate according to any convenient format, where representative formats include, but are not limited to: a divided format in which each group is present on a different region of the surface; a random format, in which the members of each group are distributed randomly on the surface, etc.; a paired format, in which probes/features for similar or analogous analytes for each species are positioned in the same region of the surface; etc.

The two or more species that are represented on a given array according to the subject invention may or may not have a known relationship with respect to each other, where in certain embodiments, the two or more species that are represented on a given array are known to have a relationship with each other. By relationship is meant a known interaction. Representative relationships of interest include, but are not limited to: host/parasite, host/pathogen; symbiotic species; and the like.

Any convenient collection of species may be represented on a given array, where representative species include, but are not limited to: viruses; virioids; prokaryotes, e.g. bacteria, archaea and cyanobacteria; and eukaryotes, e.g. members of the kingdom protista, such as flagellates, amoebas and their relatives, amoeboid parasites, ciliates and the like; members of the kingdom fungi, such as slime molds, acellular slime molds, cellular slime molds, water molds, true molds, conjugating fungi, sac fungi, club fungi, imperfect fungi and the like; plants, such as algae, mosses, liverworts, hornworts, club mosses, horsetails, ferns, gymnosperms and flowering plants, both monocots and dicots; and animals, including sponges, members of the phylum cnidaria, e.g. jelly fish, corals and the like, combjellies, worms, rotifers, roundworms, annelids, molluscs, arthropods, echinoderms, acorn worms, and vertebrates, including reptiles, fishes, birds, snakes, and mammals, e.g. rodents, primates, including humans, and the like.

Specific species groups, e.g., pairs or groupings, of interest for given representative arrays include, but are not limited to: agricultural species/pathogen, viral or parasite pairings, such as rice/magnaporthe, wheat/rust, potato/phytophthora infestans, tomato/Fusarium, sunflower/Sclerotinia, tobacco/tobacco mosaic virus, and the like; mammal (e.g. Human)/disease pathogen, such as: human gut/Helicobacter pylori (stomach ulcers); mucous membrane/Apthous ulcers; also, screening of common and/or esoteric kinds of tissue (example, oral) or organ-specific (example, skin) bacteria and fungal (tinea) infections that might be resistant to antibiotics, antifungal agents or other forms of chemotherapy. For example, commonly known sexually transmitted diseases: bacterial (Chlamydia, gonorrhoea and syphilis), mycotic (Candida albicans, Candida glabrata and Candida krusei) and viral infections (typically but not restricted to Cytomegalovirus, Epstein Barr virus, Herpes simplex viruses, human papillomaviruses). Many of the pathogenic genomes have already/are currently being sequenced. From both a research and diagnostic perspective, a duplex or multiplexed species array would potentially be very informative. Other species as well: Mouse/murine pathogens (e.g. Sendai virus, Pneumonia Virus of Mice, etc.) and the like. Further description of the arrays of the present invention, including representative substrate materials, densities of probes/features, configurations and the like, is provided in the Definitions section, above.

Methods of Fabrication

The subject arrays may be produced using any convenient protocol. Various methods for forming arrays from pre-formed probes or methods for generating the array using synthesis techniques to produce the probes in situ are generally known in the art. For example, probes can either be synthesized directly on the solid support or substrate to be used in the assay or attached to the substrate after they are made. Representative probe/feature production techniques are further described above in the Definitions section.

As such, the methods of fabricating the subject arrays include producing probes/feature on a surface of a solid support to produce the array, where a feature of the subject methods is that the probes/features produced on the surface(s) include two or more sets/groups each corresponding to a different species, as described above. A variety of solid supports or substrates may be used to practice the method of the invention, as listed above.

Utility

The subject arrays find use in a variety applications, where such applications are generally analyte detection applications in which the presence of a particular “target” analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g., an isotopic or fluorescent label present on the analyte, etc. The presence of the analyte in the sample is then deduced from the detection of binding complexes on the substrate surface. In many embodiments of the present methods, the same sample is contacted with the array and therefore simultaneously screened by the array for targets specific to the two or more species represented on the array.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g., a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

Where the arrays are arrays of peptide binding agents, e.g., protein arrays, specific applications of interest include analyte detection/proteomics applications, including those described in U.S. Pat. Nos.: 4,591,570; 5,171,695; 5,436,170; 5,486,452; 5,532,128 and 6,197,599 as well as published PCT application Nos. WO 99/39210; WO 00/04832; WO 00/04389; WO 00/04390; WO 00/54046; WO 00/63701; WO 01/14425 and WO 01/40803—the disclosures of which are herein incorporated by reference.

Because the arrays of the present invention includes groups or sets of probe molecules for target analytes of two or more species, the present arrays find particular use in applications where the interaction of the two or more species represented on the array is to be assayed or evaulated. For example, when using an array having probes to a host and known pathogen thereof, one can evaluate in a single assay the interaction of one or more pairs of host/pathogen genes, such as how expression of pathogen gene impacts expression of a host gene, etc. In certain embodiments, one can tailor the array to focus only on genes/proteins or other biomolecules of interest, e.g., those known to have or suspected of having an interaction. In certain applications, one can detect the amount of one species as background when studying another species (e.g., how much rice RNA contamination is present in a Magnoporthe sample being assayed). In certain applications, one can assess the degree of pathogen infection spread, e.g., by assaying various tissue samples from different regions of a host organism. The above described applications are merely representative the numerous different applications for which the subject arrays and methods of use are suited. In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in using an array made by the method of the present invention, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER device available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

Kits

Kits for use in analyte detection assays are also provided. The kits at least include the arrays of the invention, as described above. The kits may further include one or more additional components necessary for carrying out an analyte detection assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the array assay devices for carrying out an array based assay.

Such kits also typically include instructions for use in practicing array based assays. The instructions of the above described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

The following examples are offered by way of illustration and not by way of limitation.

### Experimental

Magnaporthe is an economic disease of rice and an important filamentous fungi model organism. Magnaporthe grisea is also a model system for the understanding of fungal-plant interactions. The Magnaporthe grisea (Magnaporthe POD) Microarray is fabricated by an In-Situ Synthesis process where 60-mer oligonucleotide probes are constructed one base at a time using phosphoramidite chemistry. Pulse-jet Technology is used to precisely deposit reagents across a wafer during each base addition (layer). After synthesis, the wafer is singulated into individual arrays. Each array contains 13,666 60-mer Magnaporthe oligonucleotide probes. There are also 7834 60-mer, disease-specific oligonucleotide probes to in-silico Rice leaf EST sequences, representing both subsets of transcripts that are altered in their transcription pattern during Rice Blast infection, and control transcripts whose level of transcription is unaltered during infection. There is an extensive literature on both the biology of this fungus, and the molecular and genetic interactions of Magnaporthe grisea with its host Oryza sativa L (rice). Furthermore, with the annotation of the rice genome completed, it is now possible to perform a genome-wide scanning approach to the interaction of host and pathogen, encompassing infection-related development, disease symptom production, host metabolic responses and host resistance. This type of microarray enables researchers to simultaneously characterize expression of many thousands of Magnaporthe genes and identify gene activity during important biological processes such as infection, fungicide mode-of-action, and fungicide resistance. It is also possible to measure the responses of a subset of rice genes and potentially all of the pathogen transcriptional responses in infected rice leaves.

It is evident from the above results and discussion that the present invention provides a number of advantages over previous employed methods of evaluating the interactions of two or more species as the molecular level. For example, one can observe the interaction of two or more species using a single sample. Furthermore, use of a single array with a single sample results in less work and less variability in the results obtained from the assay. Additional advantages, such as the ability to tailor an assay to focus on analytes of interest, are further described above. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior-invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. An array comprising test biopolymeric probes for targets of two or more different species, wherein said test biopolymeric probes are immobilized on a surface of a solid support.

2. The array according to claim 1, wherein said test biopolymeric probes are nucleic acid probes.

3. The array according to claim 1, wherein said test biopolymeric probes are peptide probes.

4. The array according to claim 1, wherein said array comprises a single solid support.

5. The array according to claim 1, wherein said array comprises a plurality of distinct solid supports.

6. The array according to claim 1, wherein said array comprises test biopolymeric probes to a first species and test biopolymeric probes to a second species, wherein the number ratio of probes to first species compared to second species is at least about 1/10.

7. The array according to claim 1, wherein said array comprises test biopolymeric probes to a first species and test biopolymeric probes to a second species, wherein said test biopolymeric probes from said second species are for targets whose transcription or translation pattern is known to be altered in response to the presence of said first species.

8. The array according to claim 7, wherein said first species is a parasite of said second species.

9. The array according to claim 7, wherein said first species is a pathogen of said second species.

10. The array according to claim 1, wherein said test biopolymeric probes are covalently attached to said surface of said substrate.

11. A method of preparing an array, said method comprising:
immobilizing test biopolymeric probes for targets of a first species on a surface of a solid support; and
immobilizing test biopolymeric probes for targets of a second species different from said first species on a surface of a solid support;
to prepare said array.

12. The method according to claim 11, wherein said immobilizing comprises depositing premade test biopolymeric probes onto said surface of a solid support.

13. The method according to claim 11, wherein said immobilizing comprises in situ synthesis of test biopolymeric probes on said surface of a solid support.

14. The method according to claim 11, wherein the number ratio of probes to first species compared to second species for said produced array is at least about 1/10.

15. The method according to claim 11, wherein said test biopolymeric probes from said second species are for targets whose transcription or translation pattern is known to be altered in response to the presence of said first species.

16. The method according to claim 15, wherein said first species is a parasite of said second species.

17. The method according to claim 15, wherein said first species is a pathogen of said second species.

18. A method of detecting the presence of a target in a sample, said method comprising:
(a) contacting an array according to claim 1 having a test biopolymeric probe that specifically binds to said target with a sample suspected of comprising said target under conditions sufficient for binding of said target to said test biopolymeric probe on said array to occur; and
(b) detecting the presence of binding complexes on the surface of said array to detect the presence of said target in said sample.

19. The method according to claim 18, wherein said test probe and analyte are nucleic acids.

20. The method according to claim 18, wherein said test probe and analyte are peptides.

21. The method comprising transmitting data from a method of claim 18 from a first location to a second location.

22. The method according to claim 21, wherein said second location is a remote location.

23. A method comprising receiving a transmitted result of a reading of an array obtained according to the method claim 18.

24. A kit for use in an analyte detection assay, said kit comprising:
an array according to claim 1; and
instructions for using said array in an analyte detection assay.

25. The kit according to claim 24, wherein said kit further comprises means for generating targets from two or more different species.

26. A method comprising reading an array according to claim 1 that has been contacted by a sample.
